(19) United States
(12) Patent Application Publication
(10) Pub. No.: US 2021/0353395 A1
(43) Pub. Date: Nov. 18, 2021

(54) SUCTION SYSTEM

(71) Applicants: Pilseong Kim, Los Angeles, CA (US); Tae Hoon Kim, Santa Monica, CA (US)

(72) Inventors: Pilseong Kim, Los Angeles, CA (US); Tae Hoon Kim, Santa Monica, CA (US)

(21) Appl. No.: 17/322,813

(22) Filed: May 17, 2021

Related U.S. Application Data
(60) Provisional application No. 63/026,271, filed on May 18, 2020.

(51) Int. Cl. A61C 17/06; A61B 90/00

(52) U.S. Cl. CPC A61C 17/06; A61B 90/05

(57) ABSTRACT

A suction system includes a cup-shaped High Vacuum Evacuation (HVE) device including a first body portion including a second end, the first body portion including a channel at the second end and a first through hole extending through the first body portion, and a second body portion including a first end, the second body portion including a second through hole extending through the second through hole at the first end, a shield/visor connected to the cup-shaped High Vacuum Evacuation (HVE) device at the second end of the first body portion at the channel, and a coupling configured to connect the first end of the second body portion to a nozzle, the nozzle being in fluid communication with a built-in drain line.

### CROSS-REFERENCE TO RELATED APPLICATION

This U.S. non-provisional application claims priority to and the benefit of U.S. Provisional Application No. 63/026,271 filed on May 18, 2020, the entire content of which is incorporated by reference herein.

### BACKGROUND

### 1. Field

The present disclosure relates generally to a suction system.

### 2. Description of Related Art

A variety of airborne infections may be transmitted to susceptible hosts from exposures to microorganisms released into the air via droplets. Some sources of transmission include aerosolized oral and nasal secretions from infected patients which can directly infect susceptible hosts. For example, according to the Centers for Disease Control and Prevention, droplets produced during a sneeze or cough can produce a cloud of infectious particles and aerosol which may expose hosts within six feet to twenty feet of the source. Further, indirect transmission may occur when the droplets and aerosol themselves settle on surfaces. The droplets and aerosol may dry up and leave behind residues containing potentially viable microorganisms. These microorganisms may be transported over long distance depending on the host surface and may be capable of infecting unsuspecting victims.

To combat microorganisms that spread in care facilities, some facilities convert rooms into negative-pressure rooms. However, this process can be expensive to build and maintain. In many cases, it may not be practical to convert an entire office space rented to a health care practitioner such as a dentist into a negative-pressure environment. Further, some aerosolized pathogens may spread primarily through direct transmission via droplets or may settle quickly within the room in a manner that can expose other hosts such as medical staff or visitors to the pathogen.

### SUMMARY

Aspects of one or more embodiments of the present disclosure are directed toward a suction system including a High Vacuum Evacuation (HVE) device valve suction device having a cup shape and an attached shield to assist in capturing or removing the aerosolized particles.

Aspects of one or more embodiments of the present disclosure are directed toward a suction system including a High Vacuum Evacuation (HVE) device having a cup shape and an attached shield having suction interconnects configured to couple to related art air dental suctioning systems for immediate application in dental offices.

Aspects of one or more embodiments of the present disclosure are directed toward a suction system including a cup-shaped High Vacuum Evacuation (HVE) device, a shield/visor/shield, and/or a mouth attachment arranged in a manner that does not interfere with a medical practitioner.

In an embodiment, a suction system includes a cup-shaped High Vacuum Evacuation (HVE) device including a first body portion including a second end, the first body portion including a channel at the second end and a first through hole extending through the first body portion, a second body portion including a first end, the second body portion including a second through hole extending through the second through hole at the first end, a shield/visor connected to the cup-shaped High Vacuum Evacuation (HVE) device at the second end of the first body portion at the channel, and a coupling configured to connect the first end of the second body portion to a nozzle, the nozzle being in fluid communication with a built-in drain line.

In an embodiment, the first body portion and the second body portion define an interior volume configured to receive aerosolized particles and/or fluids.

In an embodiment, the first through hole is at a first vertex of the first body portion and overlaps the second through hole.

In an embodiment, the first through hole is offset from a first vertex of the first body portion and the first vertex of the first body portion overlaps the second through hole.

In an embodiment, the first body portion further includes a vent extending through the first body portion, the vent being adjacent to the second end of the first body portion.

In an embodiment, the vent is spaced apart from the second end and extends toward the channel.

In an embodiment, the vent includes a side parallel to a portion of the second end of the first body portion.

In an embodiment, the first body portion further includes a plurality of vents including the vent, the plurality of vents being spaced apart from each other and aligned with each other along a first direction.

In an embodiment, the shield/visor includes a transparent material or a translucent material.

In an embodiment, the cup shape High Vacuum Evacuation (HVE) device includes at least one of aluminum, stainless steel, or a plastic resin.

In an embodiment, a cup-shaped High Vacuum Evacuation (HVE) device including a first body portion including a curved inner surface, a second body portion corresponding to the first body portion and overlapping the first body portion, and a coupling at an end of the second body portion configured to be coupled the second body portion to another device where the first body portion and the second body portion define an interior volume connected to a first through hole extending through the first body portion and a second through hole extending through the second body portion.

In an embodiment, the interior volume configured to receive aerosolized particles and/or fluids.

In an embodiment, the first through hole is at a first vertex of the first body portion and overlaps the second through hole.

In an embodiment, the first through hole is offset from a first vertex of the first body portion.

In an embodiment, the first body portion further includes a vent extending through the first body portion, the vent being adjacent to the second end of the first body portion.

In an embodiment, the vent is spaced apart from the second end of the first body portion.

In an embodiment, the vent includes a side parallel to a portion of a second end of the first body portion.

In an embodiment, the first body portion further includes a plurality of vents including the vent.

In an embodiment, the plurality of vents are spaced apart from each other and aligned with each other along a first direction.

In an embodiment, the cup-shaped High Vacuum Evacuation (HVE) device includes at least one of aluminum, stainless steel, or a plastic resin.

This summary is provided to introduce a selection of features and concepts of example embodiments of the present disclosure that are further described below in the detailed description. This summary is not intended to identify key or essential features of the claimed subject matter nor is it intended to be used in limiting the scope of the claimed subject matter. One or more of the described features according to one or more embodiments may be combined with one or more other described features according to one or more embodiments to provide a workable method or device.

### BRIEF DESCRIPTION OF THE DRAWINGS

FIG. 1 is a schematic diagram of vacuum system according to one or more embodiments of the present disclosure;

FIGS. 2A-2B show top views of a shield/visor prior to and after coupling to a cup-shaped High Vacuum Evacuation (HVE) device according to one or more embodiments of the present disclosure;

FIGS. 2C-2D show top views of a shield/visor and a cup-shaped High Vacuum Evacuation (HVE) device according to one or more embodiments of the present disclosure;

FIG. 3A is a perspective view of a suction system according to one or more embodiments of the present disclosure;

FIG. 3B is a side view of a suction system according to one or more embodiments of the present disclosure;

FIG. 4A-4C are side views of a dental suction system according to one or more embodiments of the present disclosure;

FIG. 5A-5D show different views of a cup-shaped High Vacuum Evacuation (HVE) device according to one or more embodiments of the present disclosure;

FIG. 6 is a perspective view showing the flow direction of aerosolized particles and other particles according to one or more embodiments of the present disclosure;

FIGS. 7A-7D show different views of a cup-shaped High Vacuum Evacuation (HVE) device according to one or more embodiments of the present disclosure;

FIGS. 8A-8E show different views of a cup-shaped High Vacuum Evacuation (HVE) device according to one or more embodiments of the present disclosure;

FIG. 9 is a top view of a cup-shaped High Vacuum Evacuation (HVE) device according to one or more embodiments of the present disclosure;

FIG. 10 is a top view of a cup-shaped High Vacuum Evacuation (HVE) device according to one or more embodiments of the present disclosure;

FIG. 11 shows a dental suction system according to one or more embodiments of the present disclosure;

FIGS. 12A-12C are different views of a mouth attachment and a cup-shaped High Vacuum Evacuation (HVE) device according to one or more embodiments of the present disclosure; and

FIG. 12D is a perspective view of the mouth attachment of FIG. 12C according to one or more embodiments of the present disclosure.

### DETAILED DESCRIPTION

The detailed description set forth below in connection with the appended drawings is intended as a description of example embodiments of the present disclosure and is not intended to represent the only forms in which the present disclosure may be embodied. The description sets forth aspects and features of the present disclosure in connection with the illustrated embodiments. It is to be understood, however, that the same or equivalent aspects and features may be accomplished by different embodiments, and such other embodiments are encompassed within the spirit and scope of the present disclosure. As noted elsewhere herein, like element numbers in the description and the drawings are intended to indicate like elements. Further, descriptions of features, configurations, and/or other aspects within each embodiment should typically be considered as available for other similar features, configurations, and/or aspects in other embodiments.

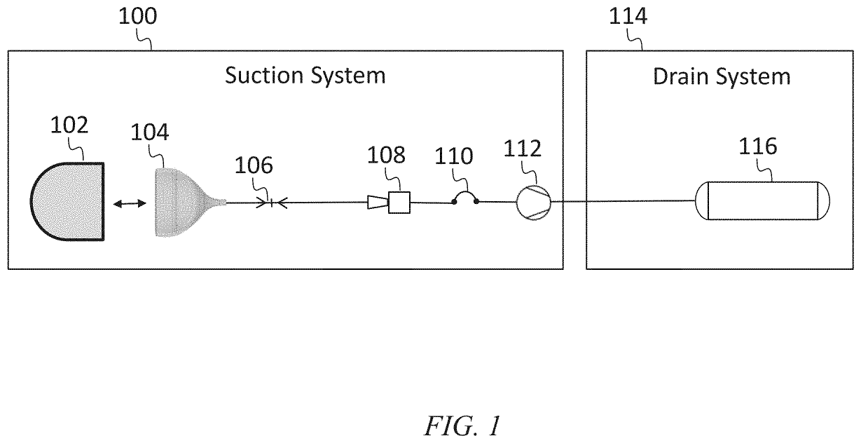

Referring to FIG. 1, a suction system 100 according to one embodiment of the present disclosure includes a shield/visor (or face shield) 102 removable attached to a cup-shaped High Vacuum Evacuation (HVE) device 104, a coupling 106 connecting an end of the cup-shaped High Vacuum Evacuation (HVE) device 104 to an end of a nozzle 108, and a hose 110 connecting the nozzle 108 to a vacuum system 112. The vacuum system 112 may be connected to a drain system 114 including a container 116 or any other suitable drainage site such as a reservoir.

In one or more embodiments, the drain system 114 may be mobile (e.g., a mobile platform including a container 116 on wheels) or may be immobile (e.g., built-in drain lines leading a reservoir or any other suitable drainage site). The drain system 114 may include components for filtering, sterilizing, and/or removing dust, dirt, and/or pathogens. For example, the drain system 114 may include filters, ultraviolet radiation systems, plasma sterilization systems, heat sterilization systems, and other treatment systems suitable for protecting the drain system 114 from damage and sterilizing aerosolized and/or other particles.

In one or more embodiments, the suction system 100 includes any suitable vacuum system 112 to remove air, dirt, debris, and aerosolized particle. The vacuum system 112 may be configured to draw in air and particles from the nozzle 108 through the hose 110 to the drain system 114 via an electrically powered motor. However, the vacuum system 112 is not limited to suction powered by electrical power and may provide suction according to any other suitable mechanism and energy source.

Additionally, in one or more embodiments, the hose 110 attached to the nozzle 108 is a flexible hose that enables a user to freely position the nozzle 108 and rigid components attached to the nozzle 108. For example, a user can grasp a body of the nozzle 108 to reposition the nozzle 108 in addition to the shield/visor 102 and a cup-shaped High Vacuum Evacuation (HVE) device 104 as desired. The nozzle 108 may include a rigid body defining a through-hole extending through the body to allow air and/or fluid flow through the body of the nozzle 108. The body has a first end and a second end with the first end connected to the hose 110 and the second end configured to be coupled to a cup-shaped High Vacuum Evacuation (HVE) device 104 via coupling 106.

In one or more embodiments, the coupling 106 may be any suitable connector configured to enable connection and disconnection between the cup-shaped High Vacuum Evacuation (HVE) device 104 and the nozzle 108. For example, the coupling 106 may connect via corresponding male-female molded structures, a twist-fit, a snap-fit, a friction fit, magnet, adhesive, and/or the like. The coupling may create an air-tight seal between a first end of the cup-shaped High Vacuum Evacuation (HVE) device 104 and the second end of the nozzle 108 to prevent leakage of fluids, particles, and air from the seal between the nozzle 108 and the cup-shaped High Vacuum Evacuation (HVE) device 104.

In one or more embodiments, the cup-shaped High Vacuum Evacuation (HVE) device 104 may extend from a narrow first end releasably coupled to the nozzle 108 via the coupling 106 to an open-faced second end. The cup-shaped High Vacuum Evacuation (HVE) device 104 may be a cup, bowl, or scoop shape having two open ends (e.g., the first end and the second end). The second end of the cup-shaped High Vacuum Evacuation (HVE) device 104 may include a rim defining a larger opening than a rim defined by the first end of the cup-shaped High Vacuum Evacuation (HVE) device 104. The first end and the second end may be at opposite ends of the cup-shaped High Vacuum Evacuation (HVE) device 104 and function as an inlet and outlet port for the cup-shaped High Vacuum Evacuation (HVE) device 104 such that aerosolized particles, fluids, and/or air can travel through the cup-shaped High Vacuum Evacuation (HVE) device 104 to the nozzle 108.

In one or more embodiments, the second end of the cup-shaped High Vacuum Evacuation (HVE) device 104 may be detachably connected to a shield/visor 102. The cup-shaped High Vacuum Evacuation (HVE) device 104 may be detachably connected to the shield/visor 102 via corresponding male-female molded structures, a twist-fit, a snap-fit, a friction fit, magnet, adhesive, and/or the like.

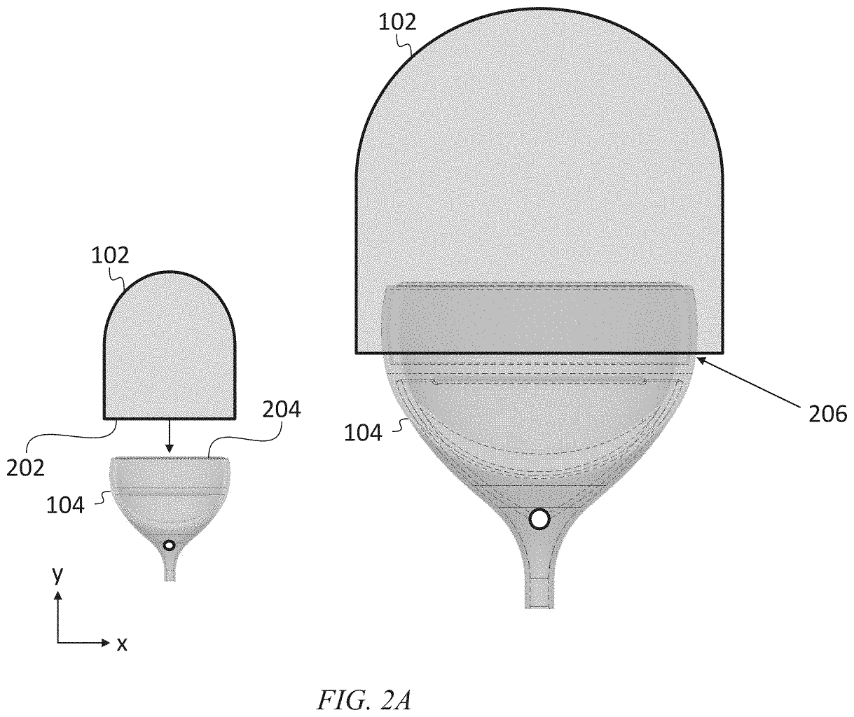

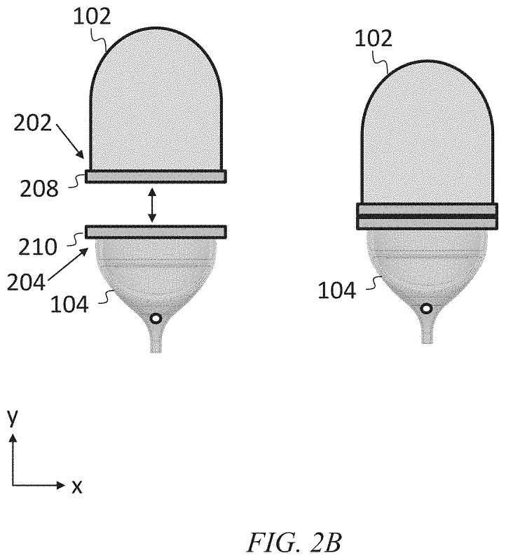

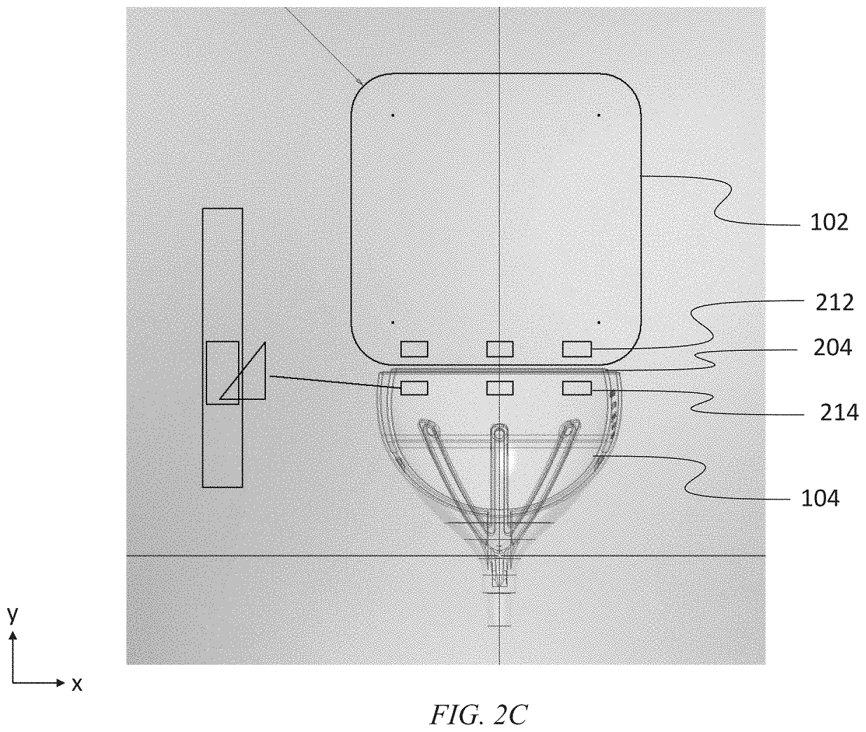

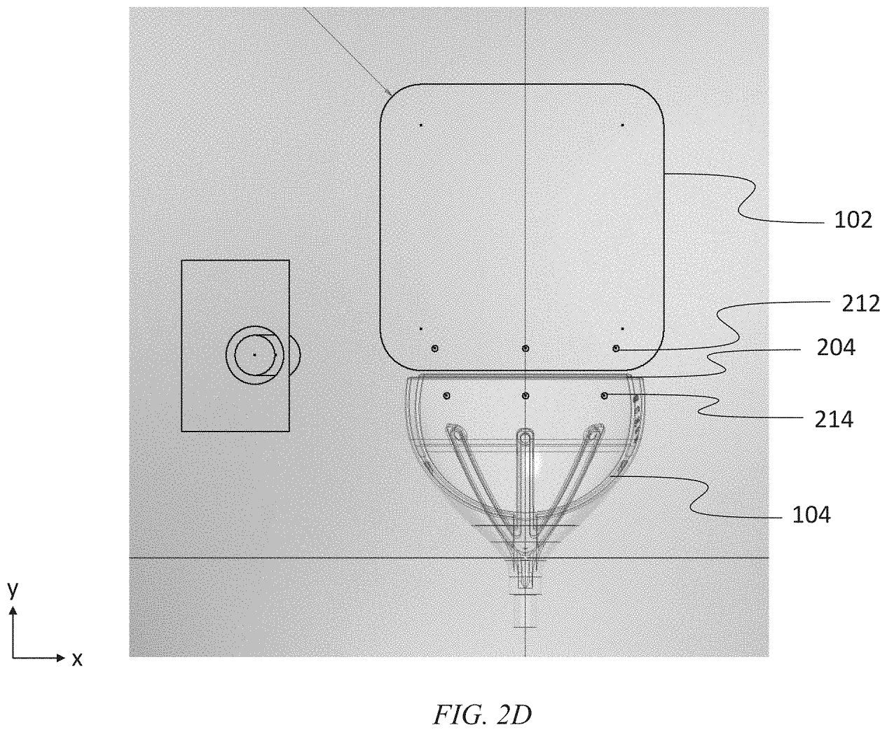

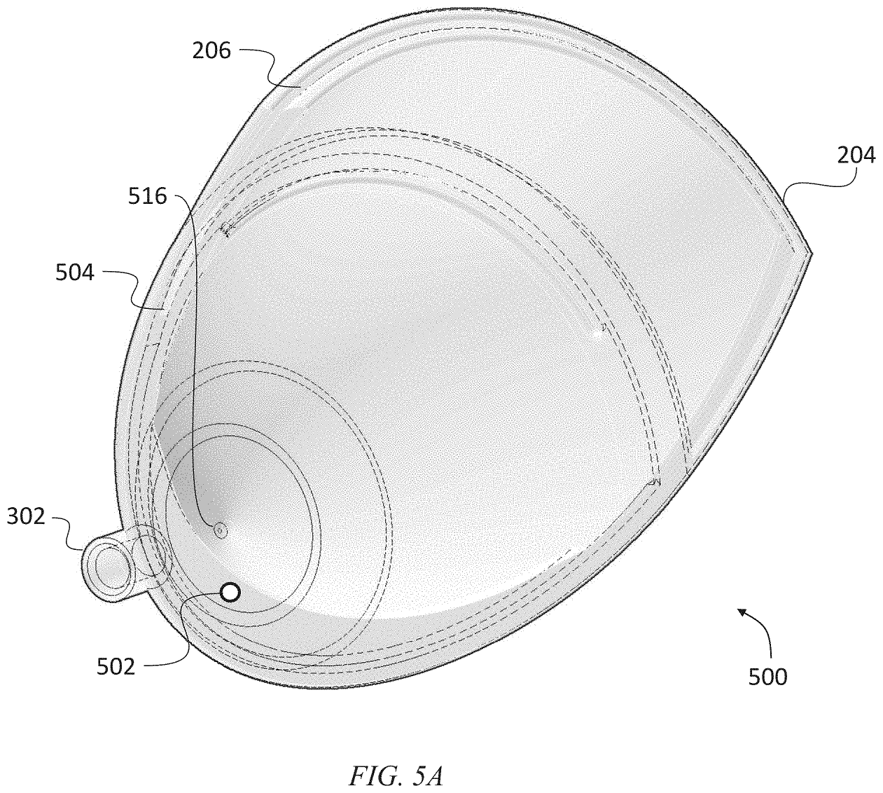

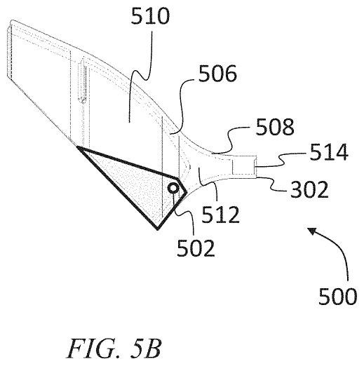

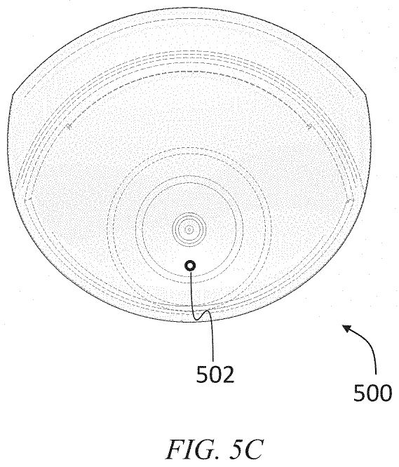

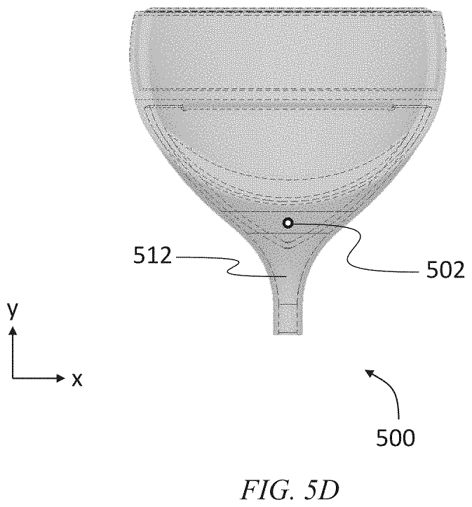

For example, as shown in FIG. 2A, a shield/visor 102 may be shaped to slide into a channel 206, slot, or trench at the second end 204 of the cup-shaped High Vacuum Evacuation (HVE) device 104 (e.g., as shown in FIG. 5A) for receiving the shield/visor 102. In the illustrated embodiment, a user may slide a side portion 202 of the shield/visor 102 (as indicated by an arrow pointing in the -y direction in FIG. 2A) into the channel 206 such that the side portion 202 of the shield/visor 102 contacts (e.g., directly contacts) a bottom of the channel 206. In the illustrated embodiment, the sidewalls of the channel 206 may grip or sandwich the shield/visor 102 to form a friction fit such that the shield/visor 102 is securely or fixedly attached to the cup-shaped High Vacuum Evacuation (HVE) device 104.

In one or more embodiments, the channel 206 may form a U-shaped channel 206 and extend along the entire rim defined by the second end 204 of the cup-shaped High Vacuum Evacuation (HVE) device 104 (e.g., as shown in the embodiment of FIG. 5A). In one or more embodiments, the channel 206 is substituted with a slot or insert that does not extend across the entire rim defined by the second end 204 of the cup-shaped High Vacuum Evacuation (HVE) device 104. In other words, the slot or insert defines a single opening overlapping a bottom of the slot or insert.

In one or more embodiments, the shield/visor 102 may include a transparent or partially transparent (i.e., translucent) material to allow a medical practitioner to view a patient's face (e.g., the mouth of the patient). The shield/visor 102 may be between the face of a patient and the face of a medical practitioner to prevent or substantially prevent aerosolized particles released from a patients nose and/or mouth from reaching the medical practitioner. Therefore, the shield/visor 102 may extend across the entire face of the patient. Although the shield/visor 102 is depicted and described in a particular manner, one or more embodiments of the present disclosure are not limited to a particular size and/or shape. The shield/visor 102 may vary in size and/or shape depending on the needs of the medical practitioner.

In one or more embodiments, the cup-shaped High Vacuum Evacuation (HVE) device 104 and shield/visor 102 have an effective range of up to about 6 inches to about 10 inches, and therefore, may be up to about 6 inches to about 10 inches away from a patients face (e.g., the mouth or nose). Accordingly, a medical practitioner may still use hand-held tools proximate to the patient's mouth area without bumping into the cup-shaped High Vacuum Evacuation (HVE) device 104 and shield/visor 102.

Because only a side portion 202 of the shield/visor 102 attaches to the cup-shaped High Vacuum Evacuation (HVE) device 104, the shield/visor 102 may vary in shape and/or size and still be attached (e.g., via friction fit) to the cup-shaped High Vacuum Evacuation (HVE) device 104. In one or more embodiments, the shield/visor 102 extends farther than any other portion of the cup-shaped High Vacuum Evacuation (HVE) device 104 in a width direction (e.g., the −x and x direction). In one or more embodiments, the shield/visor 102 extends father than any other portion of the cup-shaped High Vacuum Evacuation (HVE) device 104 in they direction. Accordingly, the cup-shaped High Vacuum Evacuation (HVE) device 104 may accommodate shield/visors 102 of varying sizes and shapes and may be suitably varied for use with adult patients and child patients. That is, the shield/visor 102 may be formed in different sizes to fit adult patients and child patients.

As another example, as shown in FIG. 2B, a shield/visor 102 may be attached at the second end 204 of the cup-shaped High Vacuum Evacuation (HVE) device 104 via magnetic ends 208, 210 respectively attached to a side portion 202 of the shield/visor 102 and a second end 204 of the cup-shaped High Vacuum Evacuation (HVE) device 104. Although the magnetic end 210 of the cup-shaped High Vacuum Evacuation (HVE) device 104 is shown in a particular position in the illustrated embodiment, the magnetic end 210 can be at any suitable position of the cup-shaped High Vacuum Evacuation (HVE) device 104. For example, the magnetic end 210 may be in a channel 206 as described in the embodiment of FIG. 2A to further secure the shield/visor 102 to the cup-shaped High Vacuum Evacuation (HVE) device 104.

To remove the shield/visor 102, a user may grip the shield/visor 102 and detach the shield/visor 102 from the cup-shaped High Vacuum Evacuation (HVE) device 104 by pulling the shield/visor 102 in a direction away from the cup-shaped High Vacuum Evacuation (HVE) device 104 (e.g., in a y direction as shown in FIGS. 2A-2B).

As another example, as shown in FIGS. 2C-2D, a shield/visor 102 may include a first retention member 212 at one end of the shield/visor 102 and a cup-shaped High Vacuum Evacuation (HVE) device 104 may include a second retention member 214 at a second end 204 of the cup-shaped High Vacuum Evacuation (HVE) device 104. The first retention member 212 and the second retention member 214 may be configured to lock with each other. For example, as shown in FIG. 2C, one of the first retention member 212 or the second retention member 214 may have a triangular shape protrusion and the other one of the first retention member 212 or the second retention member 214 may have a recess. The triangular shape protrusion may form a friction fit with the recess to lock the first retention member 212 and the second retention member 214 together when the shield/visor 102 is inserted into the cup-shaped High Vacuum Evacuation (HVE) device 104 such that the first retention member 212 and the second retention member 214 are aligned. Although a particular first retention member 212 and the second retention member 214 are described, any suitable retention member shape may be used to form a lock or friction fit. For example, as show in FIG. 2D, one of the first retention member 212 or the second retention member 214 may be a cylindrical protrusion and the other one of the first retention member 212 or the second retention member 214 may be a hole corresponding to the shape of the cylindrical protrusion.

To remove the shield/visor 102, a user may grip the shield/visor 102 and/or a cup-shaped High Vacuum Evacuation (HVE) device 104 and detach the shield/visor 102 from the cup-shaped High Vacuum Evacuation (HVE) device 104 by pulling the shield/visor 102 in a direction away from the cup-shaped High Vacuum Evacuation (HVE) device 104 (e.g., in a y direction as shown in FIGS. 2C-2D) with suitable changes to the direction of removal based on the first retention member 212 and the second retention member 214.

In one or more embodiments, the cup-shaped High Vacuum Evacuation (HVE) device 104 includes a material capable of withstanding high temperature and/or high pressure such that the cup-shaped High Vacuum Evacuation (HVE) device 600 may be autoclaved for sterilization purposes. For example, the cup-shaped High Vacuum Evacuation (HVE) device 104 may be capable of withstanding a temperature of up to 273 Fahrenheit and a pressure of 40 psi for up to 50 cycles. In one or more embodiments, the cup-shaped High Vacuum Evacuation (HVE) device 104 includes a metal such as Aluminum, Stainless Steel, etc. an autoclavable plastic resin, and/or any other autoclavable material. Therefore, the cup-shaped High Vacuum Evacuation (HVE) device 104 may be separately sterilized in response to being detached from a shield/visor 102 and nozzle.

In one or more embodiments, the shield/visor may include a transparent or translucent material such as an anti-fog clear film. In one or more embodiments, the shield/visor 102 and the cup-shaped High Vacuum Evacuation (HVE) device 104 may be a single monolithic or integral piece (i.e., the cup-shaped High Vacuum Evacuation (HVE) device 104 and the shield/visor 102 are not detachable) and may include materials that are suitable for autoclaving. In another embodiment, the shield/visor 102 is detachable from the cup-shaped High Vacuum Evacuation (HVE) device 104 and may be sterilized separately (e.g., by autoclaving or any other suitable sterilization method).

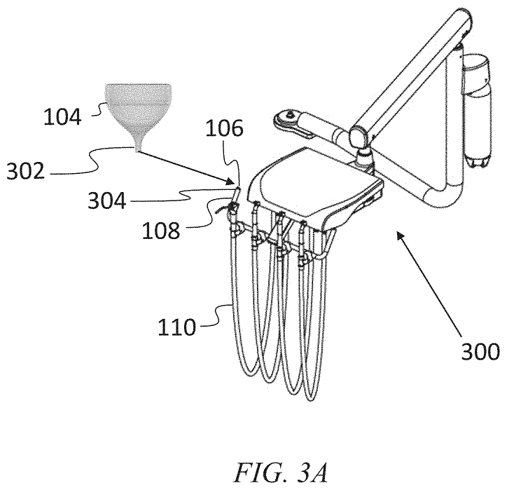

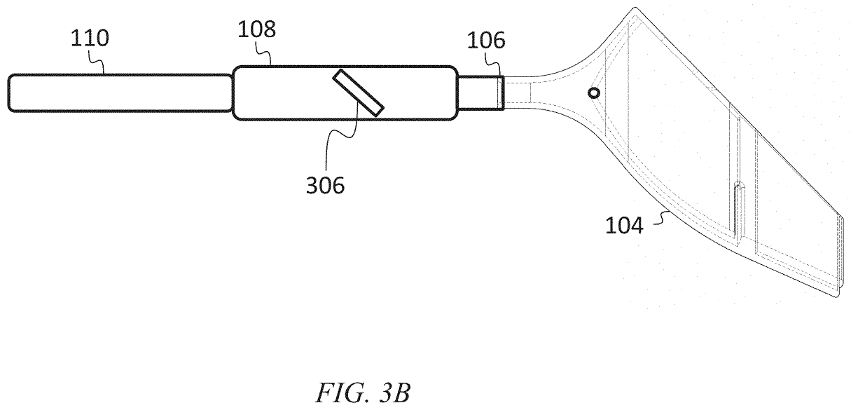

Referring to FIG. 3A, a cup-shaped High Vacuum Evacuation (HVE) device 104 may be attached to a dental suction system 300 (e.g., a related art dental suction systems such as a high vacuum evacuator (HVE) device) system via coupling 106. For example, the coupling 106 may be an 11 mm snap-in coupling that connects the cup-shaped High Vacuum Evacuation (HVE) device 104 to an HVE. Although a particular coupling 106 is described, the coupling 106 may be any suitable connector configured to enable connection and disconnection between a first end 302 of the cup-shaped High Vacuum Evacuation (HVE) device 104 and the nozzle 108. In one or more embodiments, the coupling creates an air-tight seal between a first end 302 of the cup-shaped High Vacuum Evacuation (HVE) device 104 and the second end 304 of the nozzle 108 to prevent leakage of fluids, particles, and air from the seal between the nozzle 108 and the cup-shaped High Vacuum Evacuation (HVE) device 104.

Referring to FIG. 3B, a cup-shaped High Vacuum Evacuation (HVE) device 104 may be attached to a nozzle 108 including a suction valve actuated by a switch 306. The suction valve may be actuated between a first position and a second position. The first position is an “open” position such that suction is provided with a flow direction from the cup-shaped High Vacuum Evacuation (HVE) device 104 to a hose 110. The second position is a “closed” position such that suction is cut off preventing or substantially prevent air flow from the cup-shaped High Vacuum Evacuation (HVE) device 104 to the hose 110. In one or more embodiments, the suction valve is designed to provide varying degrees of suction between the first position (i.e., the fully open position) and the second position (i.e., fully closed position), and, in other embodiments, the suction valve only has two positions. Although a suction valve actuated by a switch 306 is described, in one or more embodiments, the switch 306 may be substituted with any other device enabling and disabling suction such as a power switch that actuates a motorized pump or any other device for providing suction. In one or more embodiments, the switch 306 may be associated with multiple functions such as disabling power and closing a valve with suitable changes to the suction system 100.

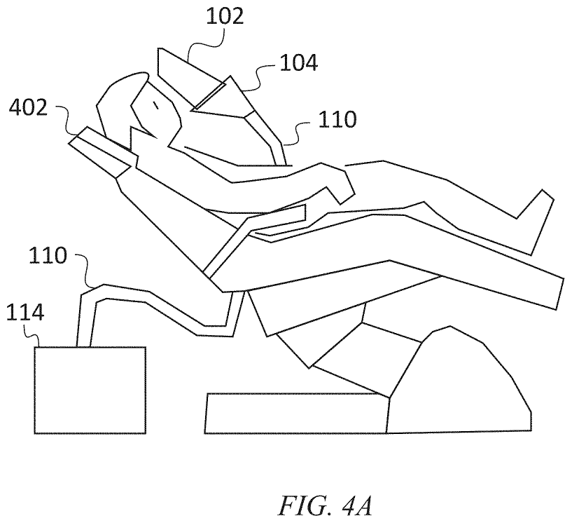

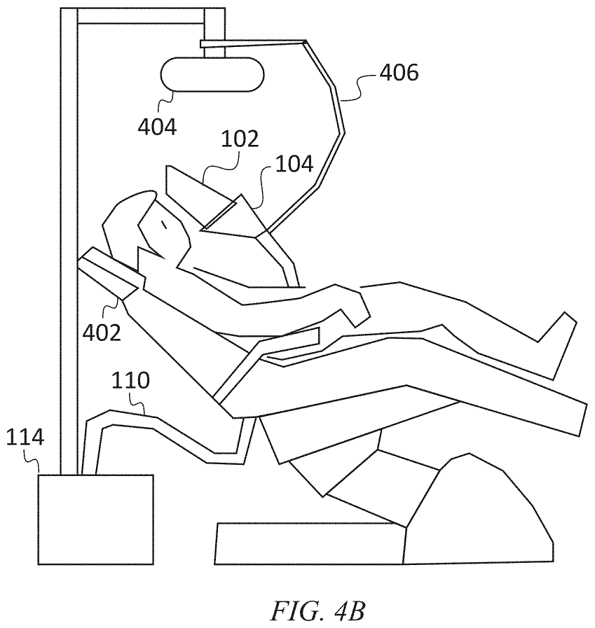

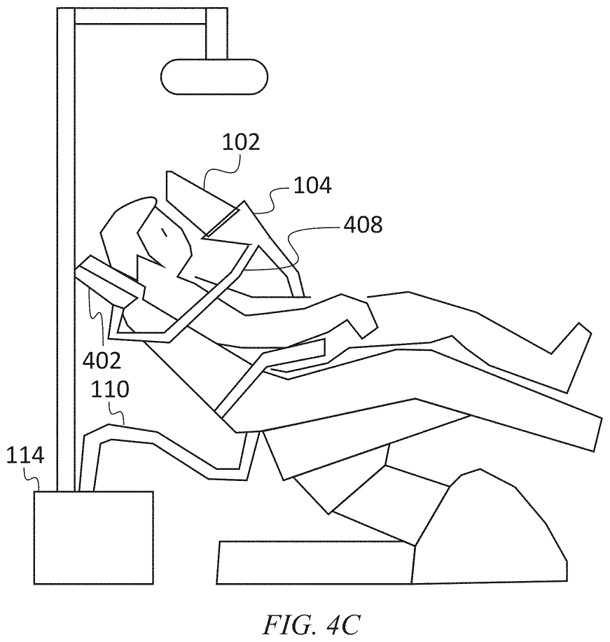

Referring to FIGS. 4A-4B, the shield/visor 102 and a cup-shaped High Vacuum Evacuation (HVE) device 104 may be positioned proximate to a patient based on a variety of different mechanisms. For example, as shown in FIG. 4A, the shield/visor 102 and the cup-shaped High Vacuum Evacuation (HVE) device 104 may be attached to a hose 110 and a drain system 114. In the illustrated embodiment, the hose 110 may be flexible such that a user holds the cup-shaped High Vacuum Evacuation (HVE) device 104 or a portion connected to the cup-shaped High Vacuum Evacuation (HVE) device 104 in order to position the shield/visor 102 and a cup-shaped High Vacuum Evacuation (HVE) device 104 proximate to the patient's face. In another embodiment, the hose 110 is rigid such that the hose 110 may hold the cup-shaped High Vacuum Evacuation (HVE) device 104 and shield/visor 102 in place without being held by a user and at the same time be capable of adjustment to a new position as desired. In another example, as shown in FIG. 4B, the cup-shaped High Vacuum Evacuation (HVE) device 104 or a portion attached to the cup-shaped High Vacuum Evacuation (HVE) device 104 may be coupled to an adjustable arm 406. A first end of the adjustable arm 406 maybe coupled to the cup-shaped High Vacuum Evacuation (HVE) device 104 or a portion attached to the cup-shaped High Vacuum Evacuation (HVE) device 104, and a second end of the adjustable arm 406 may be anchored to any nearby structure such as a light fixture 404 (e.g., a light fixture capable of emitting light (e.g., ultraviolet light) that assists in detecting aerosolized particles) above the patient. In still another example, as shown in FIG. 4C, the cup-shaped High Vacuum Evacuation (HVE) device 104 or a portion attached to the cup-shaped High Vacuum Evacuation (HVE) device 104 may be coupled to an adjustable chair arm 408. A first end of the adjustable chair arm 408 maybe coupled to the cup-shaped High Vacuum Evacuation (HVE) device 104 or a portion attached to the cup-shaped High Vacuum Evacuation (HVE) device 104, and a second end of the adjustable chair arm 408 may be anchored to a portion of a chair 402 occupied by a patient.

Referring to FIGS. 5A-5D, a cup-shaped High Vacuum Evacuation (HVE) device 500 includes a first body portion 506 and a second body portion 508 overlapping the first body portion 506 in an air/fluid flow direction. The first body portion 506 defines a first cavity 510 having a curved shape (e.g., a bowl, cup, scoop, or concave shape) to capture aerosolized particles. In one or more embodiments, the first body portion 506 includes a through hole 502 extending from the first cavity 510 to an interior volume 512 defined by the first body portion 506 and the second body portion 508. The second body portion 508 includes the first end 302 of the cup-shaped High Vacuum Evacuation (HVE) device 500 and defines a second through hole 514 extending from an external environment to the interior volume 512 at the first end 302 of the cup-shaped High Vacuum Evacuation (HVE) device 500. In one or more embodiments, the first through hole 502 and the second through hole 514 enable suction or air/fluid flow from a region proximate to the first cavity 510 to the interior volume 512 (via the first through hole 502) and the interior volume 512 to a nozzle 108 when the cup-shaped High Vacuum Evacuation (HVE) device 500 is coupled to the nozzle 108 (via coupling 106) as part of a suction system 100 including a vacuum system 112.

In one or more embodiments, the first body portion 506 has a concave shape and the second body portion 508 has a shape corresponding to the shape of the first body portion 506. The first body portion 506 may have a first vertex 516 at a lowermost portion of the first body portion 506. In one or more embodiments, the first vertex 516 may overlap the second through hole 514 as shown in FIG. 5B.

As shown in the embodiments of FIGS. 5A-5D, the first through hole 502 may be offset from the first vertex 516. In other words, the first through hole 502 may not be at the first vertex 516, and therefore, may not overlap the second through hole 514. However, the first through hole 502 extending through the first body portion 506, in one or more embodiments, may be at any suitable position. For example, the first through hole may be at the first vertex 516 as shown in the embodiments of FIGS. 2A-2B. Therefore, the first through hole 502 may align with and/or overlap the second through hole 514.

In one or more embodiments, the second body portion 508 isolates the interior volume 512 from an external environment when the cup-shaped High Vacuum Evacuation (HVE) device 500 is coupled to a nozzle 108. In other words, no vent or opening extends through the second body portion 508 such that a vent or opening connects the interior volume 512 to an external environment.

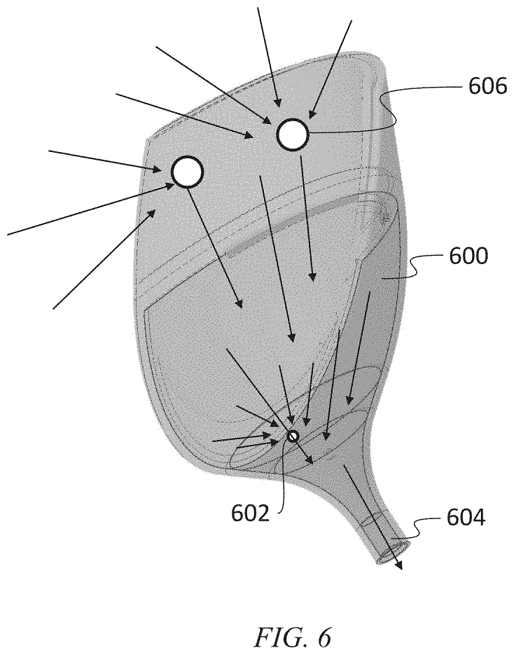

Referring to FIG. 6, a cup-shaped High Vacuum Evacuation (HVE) device 600 including a first through hole 602, a second through hole 604, and a vent 606. As shown in FIG. 6, aerosolized particles and/or water may collect on the cup-shaped High Vacuum Evacuation (HVE) device 600 before being pulled (or suctioned) toward the first through hole 602 or vent 606. The arrows in FIG. 6 indicate the flow direction of aerosolized particles and/or water toward the first through hole 602 and/or vent 606 before passing either the vent 606 and the second through hole 604 or the first through hole 602 and the second through hole 604.

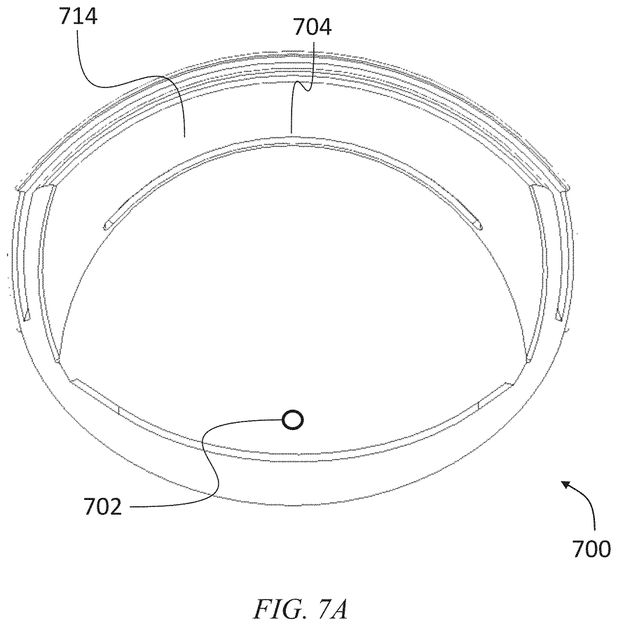

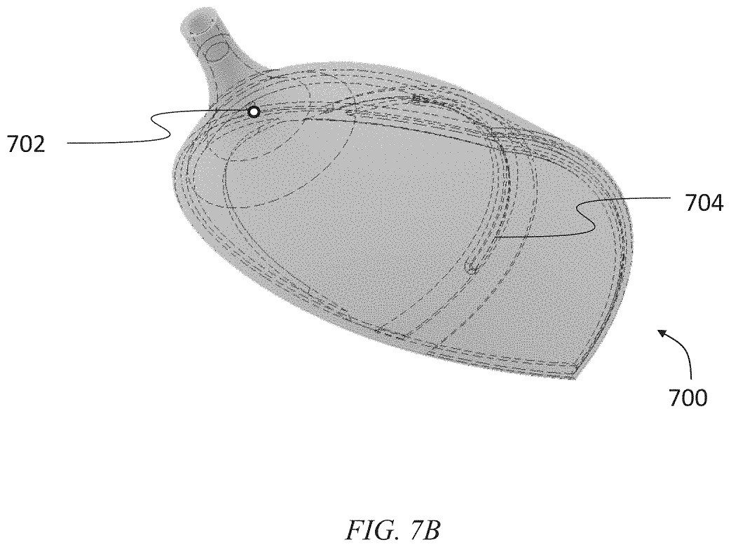

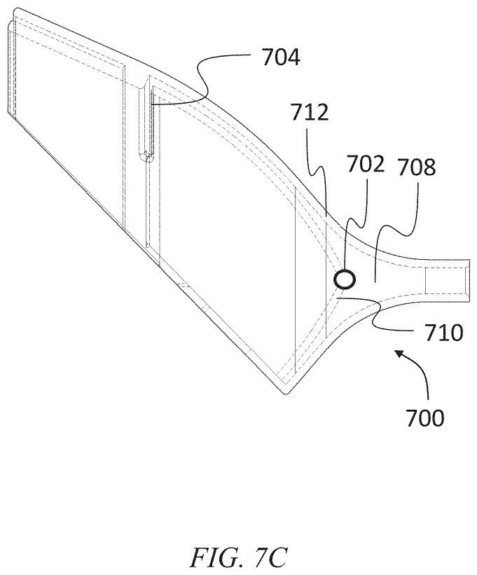

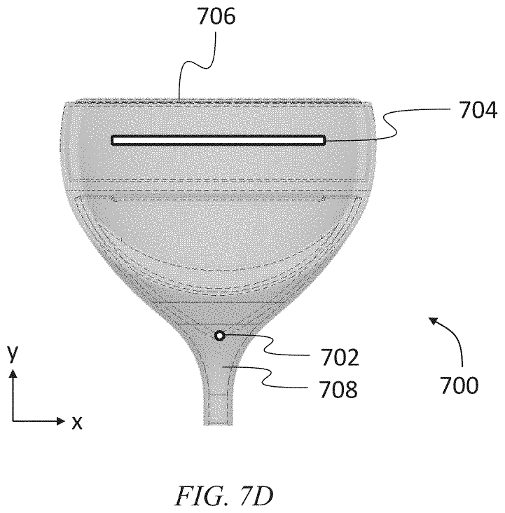

FIG. 7A is a front view of a cup-shaped High Vacuum Evacuation (HVE) device 700 according to one or more embodiments of the present disclosure. FIG. 7B is a perspective view of a cup-shaped High Vacuum Evacuation (HVE) device 700 according to one or more embodiments of the present disclosure. FIG. 7C is a side view of a cup-shaped High Vacuum Evacuation (HVE) device 700 according to one or more embodiments of the present disclosure. FIG. 7D is a top view of a cup-shaped High Vacuum Evacuation (HVE) device 700 according to one or more embodiments of the present disclosure.

Referring to FIGS. 7A-7D, a cup-shaped High Vacuum Evacuation (HVE) device 700 may include a first through hole 702 and a vent 704. The vent may be spaced apart from and adjacent to a side portion or a rim 706 of the cup-shaped High Vacuum Evacuation (HVE) device 700. In one or more embodiments, the vent may have a rectangular shape and be parallel to a side portion or a rim 706 of the cup-shaped High Vacuum Evacuation (HVE) device 700 (e.g., two sides of the vent 704 may be parallel to a side portion or a rim 706 of the cup-shaped High Vacuum Evacuation (HVE) device 700).

In one or more embodiments, the vent 704 extends through an inner surface 714 of a first body portion 710 to an interior volume 708 defined between the first body portion 710 and a second body portion 712. In other words, the vent 704 connects an external environment to the interior volume 708 such that suction applied by a suction system 100 using the cup-shaped High Vacuum Evacuation (HVE) device 700 may draw or pull aerosolized particles and/or fluid through the vent 704 to a drain system 114. Notably, the second body portion 712 does not include a through hole connecting the interior volume 708 to an external environment when a nozzle is coupled to the second body portion 712 via coupling. Therefore, aerosolized particles and/or fluid may be “sucked” or drawn from an external environment into the drain system 114 through the first through hole 702 and the vent 704.

The vent 704 may include two opposing short sides and two opposing long sides with the long sides being parallel to the portion of the second end.

Although a particular location and shape for a vent 704 and a first through hole 702 are described and shown in FIGS. 7A-7D, in one or more embodiments, different shapes and/or positions are used.

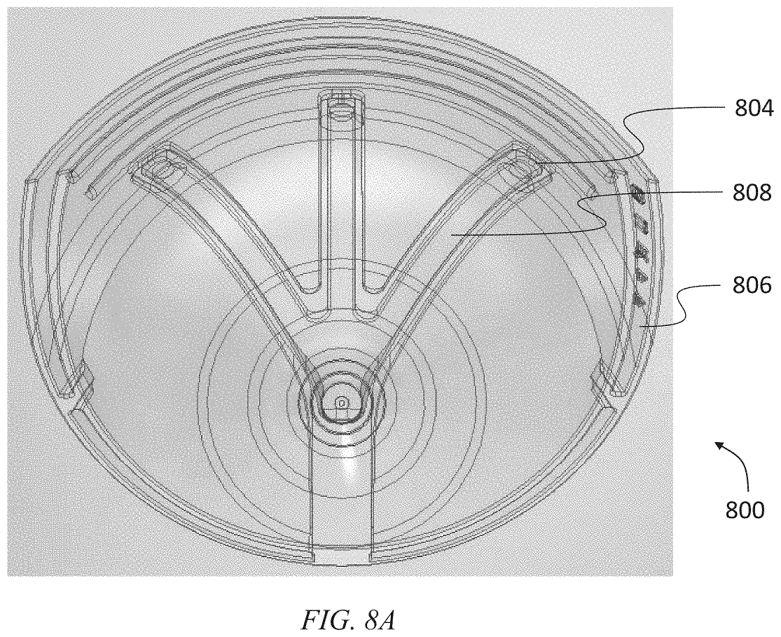

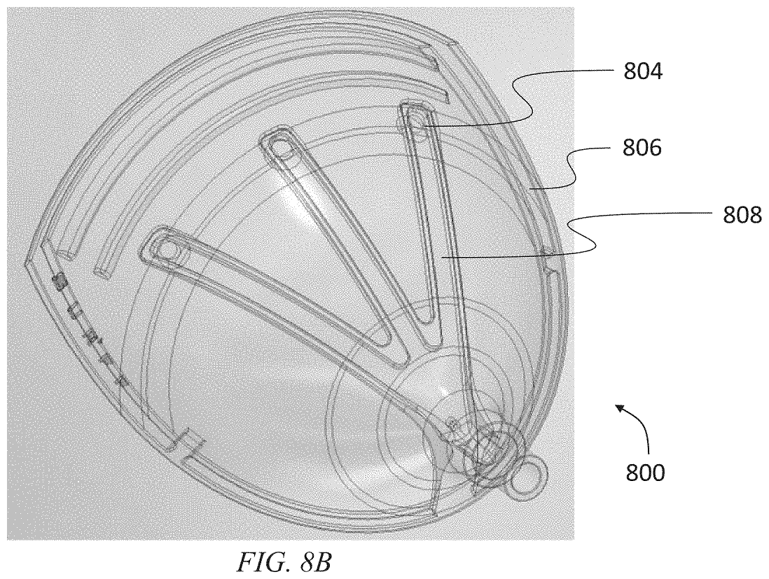

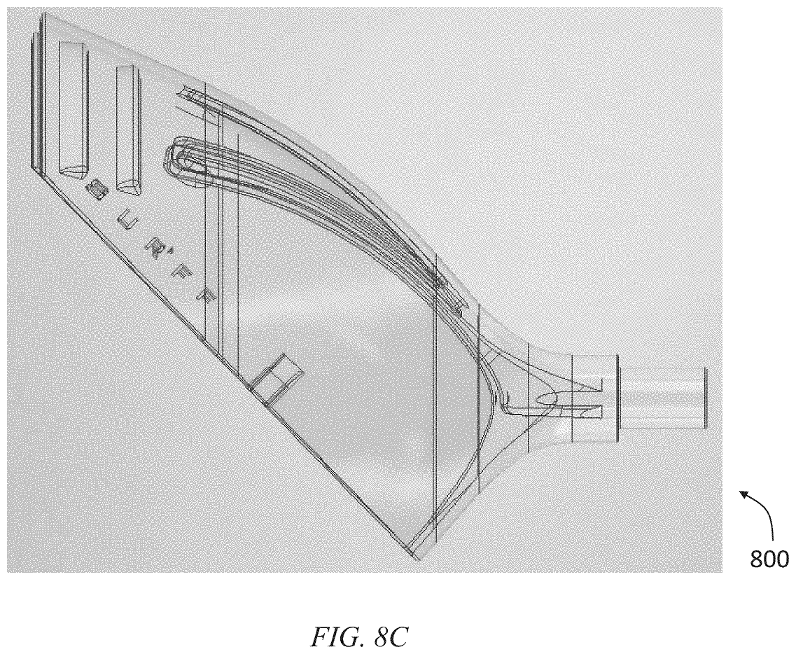

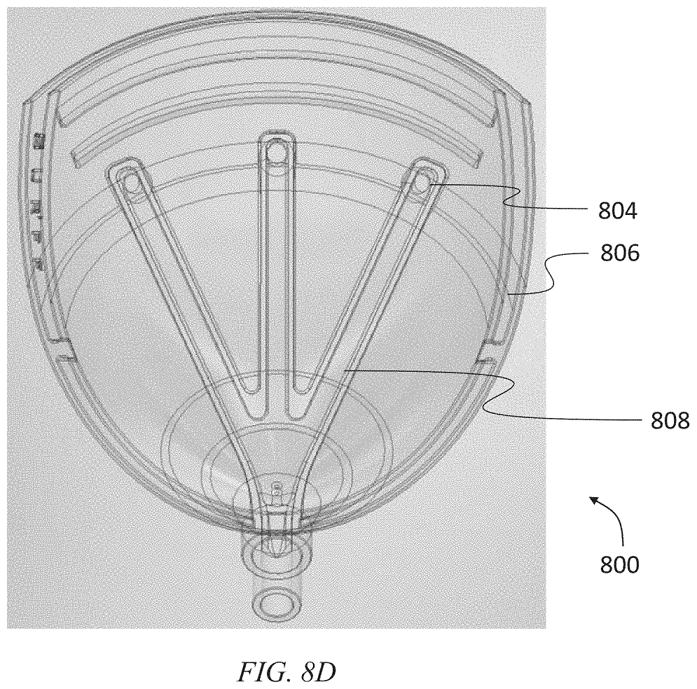

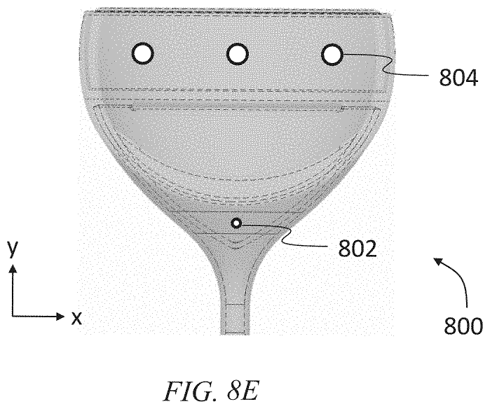

FIG. 8A is a front view of a cup-shaped High Vacuum Evacuation (HVE) device 800 according to one or more embodiments of the present disclosure. FIG. 8B is a perspective view of a cup-shaped High Vacuum Evacuation (HVE) device 800 according to one or more embodiments of the present disclosure. FIG. 8C is a side view of a cup-shaped High Vacuum Evacuation (HVE) device 800 according to one or more embodiments of the present disclosure. FIG. 8D is a top view of a cup-shaped High Vacuum Evacuation (HVE) device 800 according to one or more embodiments of the present disclosure. FIG. 8E is a top view of a cup-shaped High Vacuum Evacuation (HVE) device 800 according to one or more embodiments of the present disclosure.

With reference to FIGS. 8A-8E, a cup-shaped High Vacuum Evacuation (HVE) device 800 includes an offset first through hole 802 similar to the offset first through hole described in reference to FIGS. 5A-5D and a plurality of vents (e.g., circular or round vents) 804 extending through the first body portion of the cup-shaped High Vacuum Evacuation (HVE) device 800 in a manner similar to the vent 704.

In one or more embodiments, the vents 804 extend to a cavity defined between the first body portion of the cup-shaped High Vacuum Evacuation (HVE) device 800 and the second body portion of the cup-shaped High Vacuum Evacuation (HVE) device 800. Each vent 804 may have a corresponding cavity or vent channel 808 including a first end at the vent 804 and a second end at a vertex of the first body portion of the cup-shaped High Vacuum Evacuation (HVE) device 800. A flow direction along each of the cavities or vent channels 808 may intersect at a region proximate or adjacent to the vertex of the first body portion of the cup-shaped High Vacuum Evacuation (HVE) device 800.

In one or more embodiments, the vents 804 extend toward the channel 806 but do not contact the channel 806, and therefore, there is no air or fluid communication between the cavity or vent channel 808 and the channel 806 for securing a shield/visor.

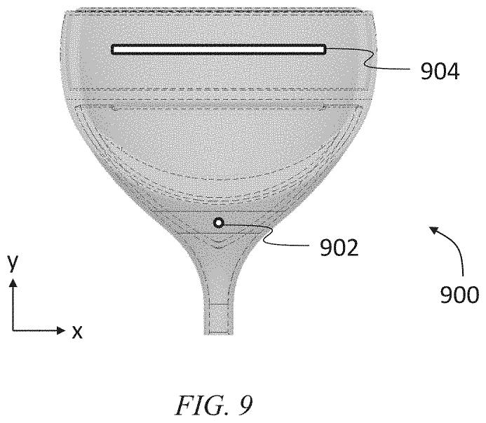

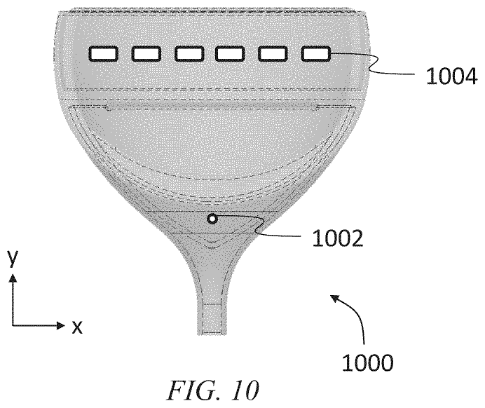

FIGS. 9-10 are top views of a cup-shaped High Vacuum Evacuation (HVE) devices 900 and 1000 according to embodiments of the present disclosure.

With reference to FIG. 9, in one or more embodiments, a cup-shaped High Vacuum Evacuation (HVE) device 900 includes an offset first through hole 902 similar to the offset first through hole described in reference to FIGS. 5A-5D and a vent 904 extending through the first body portion of the cup-shaped High Vacuum Evacuation (HVE) device 900 in a manner similar to the vent 704.

With reference to FIG. 10, in one or more embodiments, a cup-shaped High Vacuum Evacuation (HVE) device 1000 includes an offset first through hole 1002 similar to the offset first through hole described in reference to FIGS. 5A-5D and a plurality of rectangular vents 1004 extending through the first body portion of the cup-shaped High Vacuum Evacuation (HVE) device 1000 in a manner similar to the vent 704. The plurality of rectangular vents 1004 are spaced apart from each other and aligned in an x direction.

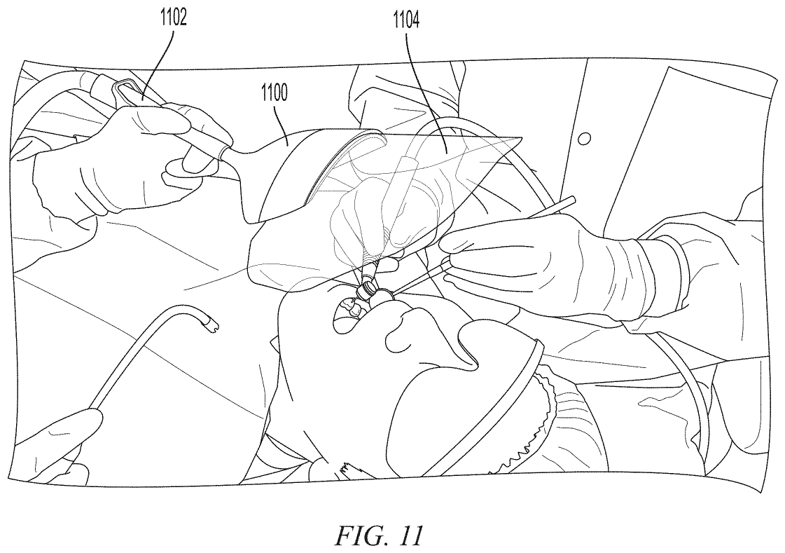

FIG. 11 shows a dental suction system according to one or more embodiments of the present disclosure.

With reference to FIG. 11, a cup-shaped High Vacuum Evacuation (HVE) device 1100 coupled to a nozzle 1102 and a shield/visor 1104 may be held proximate to a patient face (e.g., up to about 6 inches to about 10 inches away from a patient's face) such that a medical practitioner (e.g., a dentist) can perform an operation using tools at the patient's mouth area.

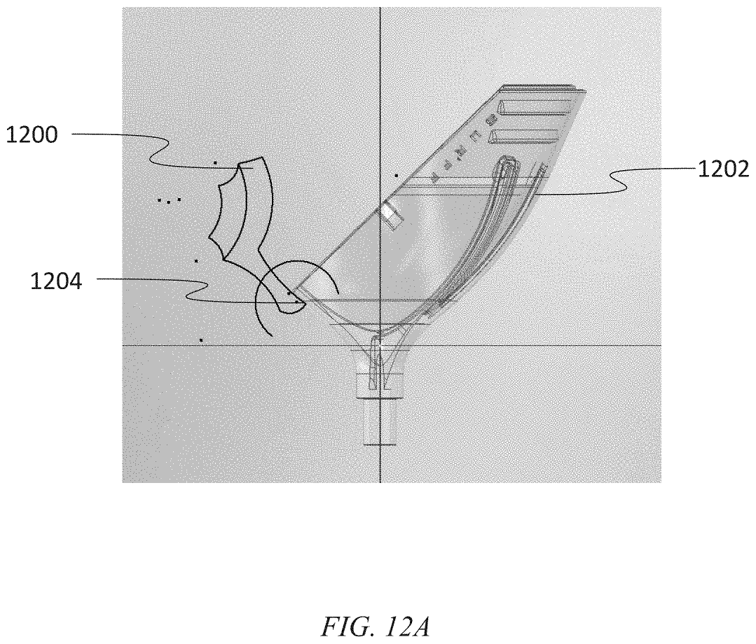

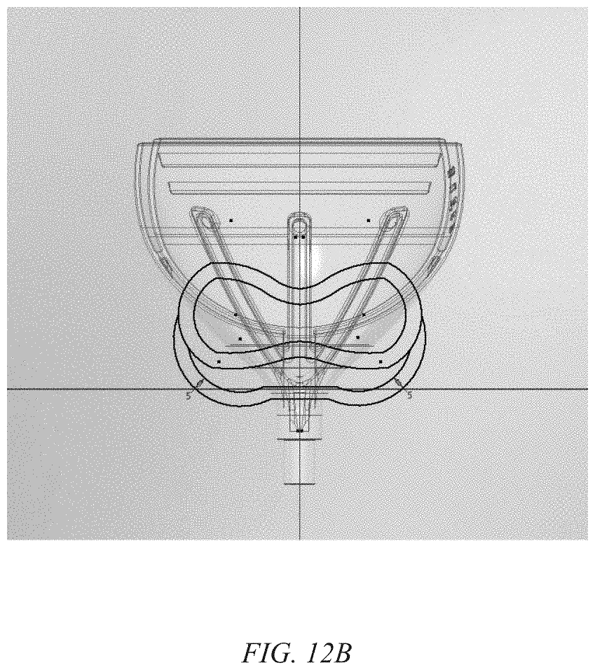

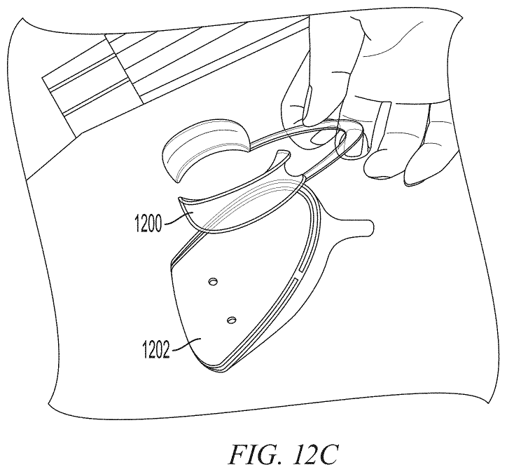

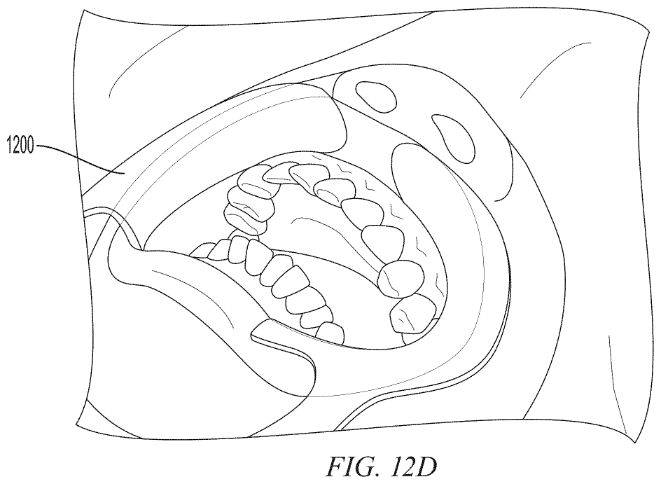

FIGS. 12A-12C are different views of a mouth attachment and a cup-shaped High Vacuum Evacuation (HVE) device according to one or more embodiments of the present disclosure; and FIG. 12D is a perspective view of the mouth attachment of FIG. 12C according to one or more embodiments of the present disclosure.

With reference to FIG. 12A-12D, in one or more embodiments, a mouth attachment 1200 may be detachably coupled to the cup-shaped High Vacuum Evacuation (HVE) device 1202 via coupling 1204 such that the mouth attachment 1200 may pivot about the coupling 1204. Therefore, the mouth attachment 1200 may remain coupled to the cup-shaped High Vacuum Evacuation (HVE) device 1202 while being adjusted to different positions.

In one or more embodiments, the mouth attachment 1200 may be used to keep a patient's mouth open as shown in FIG. 12D. The mouth attachment 1200 has suitable dimensions such that the cup-shaped High Vacuum Evacuation (HVE) device 1202 is within an effective range (e.g., about 6 inches to about 10 inches) from the patient's face in response to adjustments in the angle between the mouth attachment 1200 and the cup-shaped High Vacuum Evacuation (HVE) device 1202 when the mouth attachment 1200 is coupled to the cup-shaped High Vacuum Evacuation (HVE) device 1202.

Although any suitable coupling 1204 may be used, in one or more embodiments, the coupling 1204 may use a protrusion/recess locking mechanism similar to the first retention member and the second retention member described in FIGS. 2C-2C.

Accordingly, as disclosed herein, embodiments of the present disclosure include a suction system that assists in capturing or removing aerosolized particles released by patients to shield or protect susceptible hosts.

It will be understood that, although the terms “first”, “second”, “third”, etc., may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section, without departing from the spirit and scope of the inventive concept.

Spatially relative terms, such as “beneath”, “below”, “lower”, “under”, “above”, “upper” and the like, may be used herein for ease of description to describe one element or feature's relationship to another element(s) or feature(s) as illustrated in the figures. It will be understood that such spatially relative terms are intended to encompass different orientations of the device in use or in operation, in addition to the orientation depicted in the figures. For example, if the device in the figures is turned over, elements described as “below” or “beneath” or “under” other elements or features would then be oriented “above” the other elements or features. Thus, the example terms “below” and “under” can encompass both an orientation of above and below. The device may be otherwise oriented (e.g., rotated 90 degrees or at other orientations) and the spatially relative descriptors used herein should be interpreted accordingly. In addition, it will also be understood that when a layer is referred to as being “between” two layers, it can be the only layer between the two layers, or one or more intervening layers may also be present.

The terminology used herein is for the purpose of describing particular embodiments only and is not intended to be limiting of the inventive concept. As used herein, the terms “substantially,” “about,” and similar terms are used as terms of approximation and not as terms of degree, and are intended to account for the inherent deviations in measured or calculated values that would be recognized by those of ordinary skill in the art. As used herein, the term “major component” means a component constituting at least half, by weight, of a composition, and the term “major portion”, when applied to a plurality of items, means at least half of the items.

As used herein, the singular forms “a” and “an” are intended to include the plural forms as well, unless the context clearly indicates otherwise. It will be further understood that the terms “comprises” and/or “comprising”, when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Further, the use of “may” when describing embodiments of the inventive concept refers to “one or more embodiments of the present disclosure”. Also, the terms “exemplary” and “example” are intended to refer to an example or illustration. As used herein, the terms “use,” “using,” and “used” may be considered synonymous with the terms “utilize,” “utilizing,” and “utilized,” respectively.

It will be understood that when an element or layer is referred to as being “on”, “connected to”, “coupled to”, or “adjacent to” another element or layer, it may be directly on, connected to, coupled to, or adjacent to the other element or layer, or one or more intervening elements or layers may be present. In contrast, when an element or layer is referred to as being “directly on”, “directly connected to”, “directly coupled to”, or “immediately adjacent to” another element or layer, there are no intervening elements or layers present.

Any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this specification is intended to include all higher numerical limitations subsumed therein.

Although example embodiments of a suction system have been described and illustrated herein, many modifications and variations within those embodiments will be apparent to those skilled in the art. Accordingly, it is to be understood that a suction system according to the present disclosure may be embodied in forms other than as described herein without departing from the spirit and scope of the present disclosure.

What is claimed is:

1. A suction system comprising:
a cup-shaped High Vacuum Evacuation (HVE) device comprising:
a first body portion comprising a second end, the first body portion including a channel at the second end and a first through hole extending through the first body portion;
a second body portion comprising a first end, the second body portion including a second through hole extending through the second through hole at the first end;
a shield/visor connected to the cup-shaped High Vacuum Evacuation (HVE) device at the second end of the first body portion at the channel; and
a coupling configured to connect the first end of the second body portion to a nozzle, the nozzle being in fluid communication with a built-in drain line.

2. The suction system of claim 1, wherein the first body portion and the second body portion define an interior volume configured to receive aerosolized particles and/or fluids.

3. The suction system of claim 1, wherein the first through hole is at a first vertex of the first body portion and overlaps the second through hole.

4. The suction system of claim 1, wherein the first through hole is offset from a first vertex of the first body portion and the first vertex of the first body portion overlaps the second through hole.

5. The suction system of claim 1, wherein the first body portion further comprises a vent extending through the first body portion, the vent being adjacent to the second end of the first body portion.

6. The suction system of claim 5, wherein the vent is spaced apart from the second end and extends toward the channel.

7. The suction system of claim 5, wherein the vent includes a side parallel to a portion of the second end of the first body portion.

8. The suction system of claim 7, wherein the first body portion further comprises a plurality of vents comprising the vent, the plurality of vents being spaced apart from each other and aligned with each other along a first direction.

9. The suction system of claim 1, wherein the shield/visor comprises a transparent or a translucent material.

10. The suction system of claim 1, wherein the cup-shaped High Vacuum Evacuation (HVE) device comprises at least one of aluminum, stainless steel, or a plastic resin.

11. A cup-shaped High Vacuum Evacuation (HVE) device comprising:
a first body portion including a curved inner surface;
a second body portion corresponding to the first body portion and overlapping the first body portion; and
a coupling at an end of the second body portion configured to be coupled the second body portion to another device.
wherein the first body portion and the second body portion define an interior volume connected to a first through hole extending through the first body portion and a second through hole extending through the second body portion.

12. The cup-shaped High Vacuum Evacuation (HVE) device of claim 11, wherein the interior volume configured to receive aerosolized particles and/or fluids.

13. The cup-shaped High Vacuum Evacuation (HVE) device of claim 11, wherein the first through hole is at a first vertex of the first body portion and overlaps the second through hole.

14. The cup-shaped High Vacuum Evacuation (HVE) device of claim 11, wherein the first through hole is offset from a first vertex of the first body portion.

15. The cup-shaped High Vacuum Evacuation (HVE) device of claim 11, wherein the first body portion further comprises a vent extending through the first body portion, the vent being adjacent to a second end of the first body portion.

16. The cup-shaped High Vacuum Evacuation (HVE) device of claim 15, wherein the vent is spaced apart from the second end of the first body portion.

17. The cup-shaped High Vacuum Evacuation (HVE) device of claim 15, wherein the vent includes a side parallel to a portion of the second end of the first body portion.

18. The cup-shaped High Vacuum Evacuation (HVE) device of claim 17, wherein the first body portion further comprises a plurality of vents comprising the vent.

19. The cup-shaped High Vacuum Evacuation (HVE) device of claim 17, wherein the plurality of vents are spaced apart from each other and aligned with each other along a first direction.

20. The cup-shaped High Vacuum Evacuation (HVE) device of claim 11, wherein the cup-shaped High Vacuum Evacuation (HVE) device comprises at least one of aluminum, stainless steel, or a plastic resin.
